(19) United States
(12) Patent Application Publication
(10) Pub. No.: US 2017/0100438 A1
(43) Pub. Date: Apr. 13, 2017

(54) TREATMENT OF GLIOMA BY ANTI-ANGIOGENIC ACTIVE IMMUNIZATION FOR DIRECT TUMOR INHIBITION AND AUGMENTATION OF CHEMOTHERAPY, IMMUNOTHERAPY AND RADIOTHERAPY EFFICACY

(71) Applicant: Batu Biologics, Inc., San Diego, CA (US)

(72) Inventors: Samuel C. Wagner, San Diego, CA (US); Thomas E. Ichim, San Diego, CA (US); Santosh Kesari, San Diego, CA (US); Vladimir Bogin, San Diego, CA (US)

(21) Appl. No.: 15/288,493

(22) Filed: Oct. 7, 2016

Related U.S. Application Data
(60) Provisional application No. 62/239,222, filed on Oct. 8, 2015.

(51) Int. Cl. A61K 35/50; C12N 5/071

(52) U.S. Cl. CPC A61K 35/50; C12N 5/069; C12N 2506/025

(57) ABSTRACT

Disclosed are compositions of matter, therapeutic protocols, and immunization means to induce an active immune response to vasculature feeding glioma or other brain neoplasia. In one embodiment the invention provides administration of placental derived endothelial cells at concentrations of 10 million to 50 million administered in a manner to stimulate immunity toward blood vessels supplying glioma or other brain neoplastic malignancies. The invention provides means of blocking augmenting efficacy of immunotherapy, chemotherapy, and radiotherapy.

### CROSS REFERENCE TO RELATED APPLICATION

This application claims priority to U.S. Provisional Patent Application 62/239,222 filed on Oct. 8, 2015, the entirety of which is incorporated herein by reference.

### BACKGROUND OF THE INVENTION

Glioblastoma multiforme is the most common and most aggressive form of primary brain tumour with an incidence of 2.8 cases per 100,000 per year in the United States. Due to the highly infiltrative nature of GBM and the intrinsic chemoresistance of GBM cells, 80% of tumours recur within 2 cm of the tumour resection cavity or in the context of tumours treated by radiotherapy and chemotherapy alone, recurrence most commonly occurs adjacent to the original tumour mass. As systemic dissemination of GBM is extremely rare and the median survival for recurrent GBM is typically less than 1 year, there is a clear and rational need for effective strategies aimed at improving local tumour control.

Techniques attempted in clinical trials to improve the local control of GBM have included the direct infusion or implantation of conventional chemotherapeutic agents such as carmustine, paclitaxel and topotecan, or novel cytotoxic agents, including oncolytic herpes simplex and adenoviral vector viral and non-viral mediated gene therapy and immunotoxins such as IL13-PE38QQR, into the tumour mass, resection cavity or peritumoral tissue. To date, the only technique of localised drug delivery that has become clinically accepted is the implantation of carmustine wafers (Gliadel) into the tumour resection cavity. However, a recent Cochrane Collaboration Review of the use of Gliadel wafers concluded that in combination with radiotherapy, Gliadel has survival benefits in the management of primary disease in a “limited number” of patients, but has “no demonstrable survival benefits in patients with recurrent disease”.

Treatment for brain gliomas depends on the location, the cell type and the grade of malignancy. Histological diagnosis is mandatory, except in rare cases where biopsy or surgical resection is too dangerous. Often, treatment is a combined approach, using surgery, radiation therapy, and chemotherapy. The choice of treatments depends mainly on the histological study including the grading of the tumor. But unfortunately, the histological grading remains partly subjective and not always reproducible. Therefore, it is essential to define most relevant biological criteria to better adapt the treatments.

Blood vessels that make up the cardiovascular system may be broadly divided into arteries, veins and capillaries. Arteries carry blood away from the heart at relatively high pressure; veins carry blood back to the heart at low pressure, while capillaries provide the link between the arterial and venous blood supply. During embryonic development, vessels are first formed through vasculogenesis, utilizing pluripotent endothelial cell precursors. Later, through arteriogenesis, larger blood vessels are formed possessing a more complex structure of endothelial cells, smooth muscle cells and pericytes (tunica media). Although arteriogenesis is not considered to occur in the adult, blood vessels may be formed in the adult through vasculogenesis and notably a process known as angiogenesis. Under normal conditions, angiogenic neovascularization occurs during such conditions as wound repair, ischemic restoration and the female reproductive cycle (generating endometrium forming the corpus luteum and during pregnancy to create the placenta). The capillaries, relatively simple vessels formed by angiogenesis, lack a developed tunica as they are predominantly composed of endothelial cells and to a lesser extent perivascular cells and basement membrane.

Cancer is a disease state characterized by the uncontrolled proliferation of altered tissue cells. Tumors less than a few millimeters in size utilize nearby normal vessels to provide nutrients and oxygen. However, above this critical size, cancer cells utilize angiogenesis to create additional vascular support. Normally, angiogenesis is kept in check by the body naturally creating angiogenic inhibitors to counteract angiogenic factors. However, the cancer cell changes this balance by producing angiogenic growth factors in excess of the angiogenic inhibitors, thus favoring blood vessel growth. Cancer initiated angiogenesis is not unlike angiogenesis observed during normal vessel growth. Angiogenic factors pass from the tumor cell to the normal endothelium, binding the endothelial cell, activating it and inducing endothelial signaling events leading to endothelial cell proliferation. Endothelial tubes begin to form, homing in toward the tumor with the formation of capillary loops. Capillaries then undergo a maturation process to stabilize loop structure. Cancer is but one disease associated with a pathological neovasculature. A wide variety of diseases involving aberrant angiogenesis exist in nature. These diseases utilize the same steps involved in normal capillary growth but do so in an aberrant manner creating capillaries which lack a high degree of stability and function. Agents capable of inhibiting angiogenesis would be expected to exert activity on a variety of pathological neovascular diseases.

ValloVax, as described in Ichim et al J Transl Med 84:1443, 2015 is an endothelial derived vaccine capable of stimulating immunity against tumor endothelium. The current invention describes the use of ValloVax in treatment of glioma.

### SUMMARY OF THE INVENTION

The following presents a simplified overview of the example embodiments in order to provide a basic understanding of some aspects of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented.

Disclosed are compositions of matter, therapeutic protocols, and immunization means to induce an active immune response to vasculature feeding glioma or other brain neoplasia. In one embodiment the invention provides administration of placental derived endothelial cells at concentrations of 10 million to 50 million administered in a manner to stimulate immunity toward blood vessels supplying glioma or other brain neoplastic malignancies. The invention provides means of blocking augmenting efficacy of immunotherapy, chemotherapy, and radiotherapy.

### BRIEF DESCRIPTION OF THE DRAWING

FIG. 1 is a graph illustrating the inhibition of glioma as compared to a control.

### DETAILED DESCRIPTION OF THE PREFERRED EMBODIMENTS

The invention provides a therapeutic composition useful for stimulation of immunity against proliferating endothelium, with particular emphasis on glioma and brain neoplasms. In one specific embodiment of the invention, endothelial cells are derived from placental tissue, isolated into a homogeneous or semi-homogeneous mixture, treated with agents capable of augmenting immunogenicity, and subsequently administered into a recipient in which immune response to proliferating endothelium is desired. In one specific example, endothelial cells are purified from a human placenta according to the following steps:

a) Fetal membranes are manually peeled back and the villous tissue is isolated from the placental structure, with caution being used not to extract the deciduas or fibrous elements of the placental structure;

b) The fetal villous tissue is subsequently washed with cold saline to remove blood and scissors are used to mechanically digest the tissue into pieces as small as possible;

c) The minced tissue is then enzymatically digested. Specifically, about 25 grams of minced tissue is incubated with approximately 56 ml of liquid solution which has been pre-warmed to a temperature of 37 Celsius. Said solution comprised of Hanks Buffered Saline Solution (HBSS) supplemented with 25 mM of HEPES and containing Calcium and Magnesium, said solution containing 0.28% collagenase, 0.25% dispase, and 0.01% DNAse (added during the incubation periods as described below);

d) The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%;

e) In the first and second incubations, the incubation flask is set at an angle, and the tissue fragments are allowed to settle for approximately 1 minute, with 35 ml of the supernatant cell suspension being collected and replaced by 38 ml (after the first digestion) or 28 ml (after the second digestion) of fresh digestion solution. After the third digestion the whole supernatant is collected;

f) The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, said new born calf serum diluted at a ratio of 1 volume saline to 7 volumes of new born calf serum;

g) The pooled pellet is then resuspended in 35 ml of warm DMEM with 25 mM HEPES containing 5 mg DNase I;

h) The suspension is then mixed with 10 ml of 90% Percoll to give a final density of 1.027 g/ml and is centrifuged at 550 g for 10 minutes with the centrifuge brake off;

i) The pellet is then collected and resuspended in 15 ml of DMEM with 25 mM HEPES that is layered over a discontinuous Percoll gradient comprising of 20%-70% Percoll in 10% steps and centrifuged at 1900 g for 20 minutes;

j) The cells found at the 1.037 g/ml and 1.048 g/ml are collected utilized for the generation of a cellular vaccine product.

Said cellular vaccine product from step “j”, in a preferred embodiment is treated with an agent capable of augmenting immunogenicity. Said immunogenicity in this context refers to ability to enhance recognition by recipient immune system. In one embodiment, immunogenicity refers to enhanced expression of HLA I and/or HLA II molecules. In another embodiment, immunogenicity refers to enhanced expression of costimulatory molecules. Said costimulatory molecules are selected from a group comprising of: CD27; CD80; CD86; ICOS; OX-4; and 4-1 BB. In another embodiment, immunogenicity refers to enhanced ability to stimulate proliferation of allogeneic lymphocytes in a mixed lymphocyte reaction. Immunogenicity may be augmented by incubation with one of the lymphokine or cytokine proteins that are known in the art, or with a member of the interferon family.

In one particular embodiment, said purified endothelial cells are incubated with interferon gamma. In one particular embodiment, interferon gamma is incubated with endothelial cells, whether purified or unpurified for a period of approximately 48 hours, at a concentration of approximately 150 IU/ml. Endothelial cells may be expanded after purification as described above before treatment with agents capable of augmenting immunogenicity. For example, endothelial cells may be treated with an endothelial cell mitogen. Said endothelial cell mitogen may be any protein, polypeptide, variant or portion thereof that is capable of, directly or indirectly, inducing endothelial cell growth. Such proteins include, for example, acidic and basic fibroblast growth factors (aFGF) (GenBank Accession No. NP-149127) and bFGF (GenBank Accession No. AAA52448), vascular endothelial growth factor (VEGF) (GenBank Accession No. AAA35789 or NP-001020539), epidermal growth factor (EGF) (GenBank Accession No. NP-001954), transforming growth factor alpha (TGF-alpha) (GenBank Accession No. NP-003227) and transforming growth factor beta (TFG-beta) (GenBank Accession No. 1109243A), platelet-derived endothelial cell growth factor (PD-ECGF) (GenBank Accession No. NP-001944), platelet-derived growth factor (PDGF) (GenBank Accession No. 1109245A), tumor necrosis factor alpha (TNF-alpha) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP-000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994).

Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993). Before expansion, endothelial cells may be further purified based on expression of surface receptors using affinity-based methodologies that are known to one of skill in the art, said methodologies include magnetic activated cell sorting (MACS), cell panning, or affinity chromatography. Other methodologies such as fluorescent activated cell sorting (FACS) may also be used. Various lectins are known to have selectivity to endothelial cells, for example, Ulex europaeus agglutinin I is known to possess ability to bind to endothelial cells and endothelial progenitor cells. It is within the scope of the current invention to define “endothelial cell” as including “endothelial progenitor cell”.

The cancer vaccine formulation may be utilized in conjunction with known adjuvants in order to induce an immune response that is Th1 or Th17-like, and which will inhibit the proliferation of endothelial cells in the recipient. Such adjuvant compounds are known in the art to boost the activity of the immune system and are now under study as possible adjuvants, particularly for vaccine therapies. Some of the most commonly studied adjuvants are listed below, but many more are under development. For example, Levamisole, a drug originally used against parasitic infections, has recently been found to improve survival rates among people with colorectal cancer when used together with some chemotherapy drugs. It is often used as an immunotherapy adjuvant because it can activate T lymphocytes. Additionally, the compound has been demonstrated to induce maturation of dendritic cells, further supporting an immune modulatory role. Levamisole is now used routinely for people with some stages of colorectal cancer and is being tested in clinical trials as a treatment for other types of cancer. Additionally, it has been shown to augment efficacy of other immunotherapeutic agents such as interferon.

Aluminum hydroxide (alum) is one of the most common adjuvants used in clinical trials for cancer vaccines. It is already used in vaccines against several infectious agents, including the hepatitis B virus Bacille Calmette-Guerin (BCG) is a bacterium that is related to the bacterium that causes tuberculosis. The effect of BCG infection on the immune system makes this bacterium useful as a form of anticancer immunotherapy. BCG was one of the earliest immunotherapies used against cancer, either alone, or in combination with other therapies such as hormonal, chemotherapy or radiotherapy. It is FDA approved as a routine treatment for superficial bladder cancer. Its usefulness in other cancers as a nonspecific adjuvant is also being tested or has demonstrated therapeutic effects. Researchers are looking at injecting BCG to give an added stimuli to the immune system when using chemotherapy, radiation therapy, or other types of immunotherapy. Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells.

Incomplete Freund's Adjuvant (IFA) is given together with some experimental therapies to help stimulate the immune system and to increase the immune response to cancer vaccines, both protein and peptide in part by providing a localization factor for T cells. IFA is a liquid consisting of an emulsifier in white mineral oil. Another vaccine adjuvant useful for the present invention is interferon alpha, which has been demonstrated to augment NK cell activity, as well as to promote T cell activation and survival. QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines.

In one embodiment of the invention proliferating endothelial cells treated with an agent to stimulate immunogenicity are lysed and protein extracts are extracted and utilized as a vaccine. In some embodiments specific immunogenic peptides may be isolated for said cell lysate. In other embodiments, lyophilization of endothelial cells is performed subsequent to treatment with an agent that augments immunogenicity. In embodiments utilizing cellular extracts, various formulations may be generated. The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

In one embodiment of the invention, said ValloVax (Ichim et al, J Transl Med 2015) induces increased permeability of tumor endothelium allow for increased efficacy of chemotherapy and radiotherapy. Additionally, given that tumor endothelium expresses immune killing molecules such as Fas ligand, in one embodiment, the use of ValloVax together with immunotherapy is disclosed.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. In situations where an orally available vaccine is desirable, a tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described. Nasal sprays may be useful formulations. Preferred unit dosage formulations are those containing a single or daily dose or unit, daily sub-dose or an appropriate fraction thereof, of an active ingredient.

It will be appreciated that the therapeutic molecule can be delivered to the locus by any means appropriate for localized administration of a drug. For example, a solution of the therapeutic molecule can be injected directly to the site or can be delivered by infusion using an infusion pump. The construct, for example, also can be incorporated into an implantable device which when placed at the desired site, permits the construct to be released into the surrounding locus. The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic®. Although subcutaneous, intradermal, and intramuscular routes of administration are preferred, administration into lymphatics of the vaccine preparation is also envisioned within the scope of the current invention. Endpoints guiding the practitioner of the invention include: a) ability of the vaccine to stimulate immunity towards proliferating endothelial cells; b) ability of the vaccine to stimulate immunity towards cancer-associated molecules; and c) ability of the vaccine to stimulate immunity towards tumor cells.

In one embodiment the invention provides a means of generating a population of cells with ability to inhibit endothelial cell proliferation. In one embodiment approximately 50 ml of peripheral blood is extracted from a cancer patient and peripheral blood monoclear cells (PBMC) are isolated using the Ficoll Method. PBMC are subsequently resuspended in approximately 10 ml RPMI media with 10% fetal calf serum and allowed to adhere onto a plastic surface for 2-4 hours. The adherent cells are then cultured at 37° C. in RPMI media supplemented with 1,000 U/mL granulocyte-monocyte colony-stimulating factor and 500 U/mL IL-4. This procedure, or a procedure similar to it, can be utilized for the generation of dendritic cells. Half of the volume of the GM-CSF and IL-4 supplemented media is changed every other day. Immature DCs are harvested on day 7. In one embodiment said generated DC are treated with endothelial cell extracts isolated from placental or otherwise proliferating endothelial cells. Said extracts are added to said immature dendritic cells on day 7. Endothelial pulsed dendritic cells may be administered directly as a vaccine, or may be utilized to stimulate autologous patient T cell clones in vitro. Said T cell clones may be selected for specificity to proliferating endothelial cells.

Additionally, in some embodiments, whether for in vitro stimulation of T cells, or for direct use as a tumor vaccine, the endothelial cell pulsed dendritic cells may be further purified from culture through use of flow cytometry sorting or magnetic activated cell sorting (MACS), or may be utilized as a semi-pure population. In one embodiment DC are exposed to agents capable of stimulating maturation in vitro subsequent to pulsing with endothelial cell extracts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In another embodiment, a mixture of endothelial cells together with immature dendritic cells is used as a combination cellular vaccine. In another embodiment, endothelial cells (live or extracts or fixed) are administered in combination with dendritic cells together with activated T cells and/or NK cells. In order to activate T cells and/or NK cells in vitro, cells are cultured in media containing approximately 1000 IU/ml of interferon gamma.

Incubation with interferon gamma may be performed for the period of 1 hour to the period of 14 days. Preferably, incubation is performed for approximately 48 hours, after which T cells and/or NK cells may be further stimulated via the CD3 and CD28 receptors. One means of accomplishing this is by addition of antibodies capable of activating these receptors. In one embodiment approximately, 3 ug/ml of anti-CD3 antibody is added, together with approximately 2 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 400 u/mL IL-2. Media containing IL-2 and antibodies may be changed every two days for approximately 7-24 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or intralymphatically.

In some embodiments, endothelial cells are increased in immunogenicity by culture with T cells that are autologous or allogeneic to the donor of said endothelial cells. Said T cells may be activated by their allogeneic interaction with said endothelial cells, or may be introduced into contact with endothelial cells in an already preactivated state. In order to preactive T cells, firstly lymphocytes are collected and separation into the T cell population and cell sub-population containing a T cell can be performed, for example, by fractionation of a mononuclear cell fraction by density gradient centrifugation, or a separation means using the surface marker of the T cell as an index of detection. Subsequently, isolation based on surface markers may be performed. Examples of the surface marker include CD2, CD3, CD8 and CD4, and separation methods depending on these surface markers are known to one of skill in the art. For example, the step can be performed by mixing a carrier such as beads or a culturing flask onto which an anti-CD8 antibody has been immobilized (cell panning), with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as marker of detection and, for example, CD4 MicroBeads, Dynabeads M-450 CD4 can also be used.

In some embodiments of the invention, T regulatory cells are depleted before initiation of the culture, with the idea of “derepressing” suppressive elements within the heterogeneous T cell population. Depletion of T regulatory cells may be performed by negative selection by removing cells that express makers such as neuropilin, CD25, CD4, CD105, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma, IL-15, IL-7, IFN-alpha, IL-12, CD40L, and IL-27. From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1 alpha, MIP1 beta, CCL19, CXCL12, IP-10 and MIG.

The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes or antigen presenting cells (dendritic cell) presenting a target peptide such as a peptide derived from an endothelial cell antigen. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay such as the tetramer assay, a proliferation assay, a cytolytic cell assay, or an in vivo delayed hypersensitivity test using a recombinant endothelial cell-associated antigen or an immunogenic fragment or an antigen-derived peptide. In a preferred embodiment, the antigen derived peptides are specifically associated with proliferating endothelial cells, such as endothelial cells found in proximity to the tumor.

Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxicity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant endothelial-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay (ELISPOT), cytokine flow cytometry, a direct cytotoxicity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

Unless defined differently, all technical and scientific terms used herein have the same meanings as commonly understood by one of skill in the art to which the disclosed invention belongs. In particular, the following terms and phrases have the following meaning.

“Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

In some aspects of the invention, it will be important to overcome tolerance that already exists to proliferating self endothelial cells. Accordingly, on of skill in the art is directed towards the following description of tolerogenic processes, with the knowledge that manipulation and specific inhibition of these processes is useful in the practice of the current invention.

The argument has been made that tolerance is controlled to some extent by immature dendritic cells presenting self antigen in absence of costimulation/presence of co-inhibitors, which leads to generation of Treg cells and anergic T cells. This was demonstrated in several systems, for example, in a classical experiment Mahnke et al targeted the antigen ovalbumin to immature dendritic cells by conjugation to anti-DEC205 antibodies. It was demonstrated that antigen-specific Treg were generated, which was dependent on presentation by immature dendritic cells. In vivo relevance of Treg generated by targeting antigen to steady state dendritic cells can be seen in studies where DEC-205 targeting of antigen prevented autoimmune diabetes in a transgenic model system via FoxP3 expressing Treg.

We have reported on a “tolerogenic vaccine” created by ex vivo generation of immature DC treated with a chemical IKK inhibitor, and pulsed with collagen II, that was able to prevent arthritis in a mouse model. Similar tolerogenic uses of immature DC have been reported in diverse conditions such as transplantation, anti-Factor VIII immunity, autoimmune myocarditis, experimental autoimmune mysthenia gravis, and collagen induced arthritis. The possibility that tumors may be generating immature DC to protect themselves from T cell attack and/or generate Treg was suggested in studies showing tumor secreted VEGF would arrest DC maturation in vitro. Mechanistically it was demonstrated that VEGF blocks NF-kB activity in DC, which is a critical maturation-inducing factor. Given that VEGF is a primary cytokine in tumor angiogenesis, the possibility of inhibited DC maturation being a mechanism of immune escape is attractive.

Angiogenesis seems to be associated with various cells of the myeloid lineage. The myeloid suppressor cell, which will be described below, has been demonstrated stimulate angiogenesis directly, and through production of MMP-9 and VEGF. In HNSCC a population of myeloid suppressor cells was described in a series of publications by Rita Young's group. These cells, which express the hematopoietic stem cell marker CD34, were originally identified as the source of intra-tumor GM-CSF detected from primary patient samples. Suggesting a possible immune inhibitory role for these cells were data that their depletion results in upregulated ability of lymphocytes within the tumor to generate IL-2, which was lost upon re-introduction of these cells into culture. Clinical relevance of these myeloid suppressor cells was supported by a study of 20 HNSCC patients whose tumors were resected and relapsed, compared to 17 patients that had disease free survival for the 2-year observation period. Tumors of patients relapsed produced almost 4-fold higher levels of GM-CSF and had approximately 2.5-fold the number of CD34+ cells as compared to patients that were free of disease. Mechanistic study of these cells revealed suppression of T cell activity could be abolished treatment with antibodies to TGF-b, and that inhibitory activity was lost upon their differentiation with agents such as IFN-g and TNF-alpha. Given that immature DC mediate Treg generation through TGF-b, and that immature DC lose inhibitory activity upon maturation with agents such as IFN-g and TNF-alpha, the possible relationship with myeloid suppressor cells was considered. In fact, a recent study suggested the possibility of vivo differentiation of myeloid suppressor cells.

Newly diagnosed HNSCC patients were treated with Vitamin D3 for three weeks before surgical excision of the tumor. Observations of significant reduction in numbers of intratumoral CD34 cells and augmented numbers of dendritic cells were reported. Other interventions for induction of myeloid suppressor cell differentiation into DC/reversing immune suppressive potential have demonstrated some promise including 5-azacytidine, sunitinib, PDE-5 inhibitors, and inhibitors of stem cell factor or its receptor c-kit. Of these, 5-azaycytidine, sunitinib various PDE-5 inhibitors are already part of clinical practice. In the case of sunitinib, clinical evidence of depression of T cell responses after therapy has been reported, effects being mediated, in part, by suppression of STAT3 activity. Myeloid suppressor cells have been described in numerous other conditions of neoplasia, in which GM-CSF has been reported to be a major factor in their generation. In addition to TGF-beta, suppression by myeloid suppressor cells seems to be mediated by PGE-2, expression of arginase, which generates immune suppressive polyamines, and depletion of cystine and cysteine (amino acids needed for T cell activation).

Thus while it is still not completely clear how upstream in the differentiation pathway myeloid suppressor cells are as compared to immature dendritic cells, there is evidence that both cell populations mediate generation of Treg cells. In the case of HNSCC at least one paper supports in situ generation of Treg by immature antigen presenting cells, specifically, a study comparing SCC with Actinic Keratosis demonstrated that increased Treg cell numbers were associated with local DC, in SCC. Others have made correlations between myeloid suppressor cell numbers and Treg. Thus it is within the scope of the current invention to induce in vivo maturation/activation of DC, in order to augment breaking of self tolerance towards tumor tissue, with particular emphasis on tumor-associated endothelial cells.

The injection for parenteral administration of the tumor-resembling endothelial cell immunogen, otherwise termed ValloVax may be an aqueous injection or an oily injection. The aqueous injection can be prepared according to a known method, for example, by appropriately adding a pharmaceutically acceptable additive to an aqueous solvent (water for injection, purified water, etc.) to make a solution, mixing the WT1 protein or WT1 peptide with the solution, filter sterilizing the resulting mixture with a filter etc., and then filling an aseptic container with the resulting filtrate. Examples of the pharmaceutically acceptable additive include the above-mentioned adjuvants; isotonizing agents such as sodium chloride, potassium chloride, glycerol, mannitol, sorbitol, boric acid, borax, glucose and propylene glycol; buffering agents such as a phosphate buffer solution, an acetate buffer solution, a borate buffer solution, a carbonate buffer solution, a citrate buffer solution, a Tris buffer solution, a glutamate buffer solution and an epsilon-aminocaproate solution; preservatives such as methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, sodium edetate, boric acid and borax; thickeners such as hydroxyethylcellulose, hydroxypropylcellulose, polyvinyl alcohol and polyethylene glycol; stabilizers such as sodium hydrogen sulfite, sodium thiosulfate, sodium edetate, sodium citrate, ascorbic acid and dibutyl hydroxy toluene; and pH adjusters such as hydrochloric acid, sodium hydroxide, phosphoric acid and acetic acid. The injection may further contain an appropriate solubilizing agent, and examples thereof include alcohols such as ethanol; polyalcohols such as propylene glycol and polyethylene glycol; and non-ionic surfactants such as polysorbate 80, polyoxyethylene hydrogenated castor oil 50, lysolecithin and pluronic polyols. Also, proteins such as bovine serum albumin and keyhole limpet hemocyanin; polysaccharides such as aminodextran; etc. may be contained in the injection. For preparation of the oily injection, for example, sesame oil or soybean oil is used as an oily solvent, and benzyl benzoate or benzyl alcohol may be blended as a solubilizing agent. The prepared injection is usually stored in an appropriate ampule, vial, etc. The liquid preparations, such as injections, can also be deprived of moisture and preserved by cryopreservation or lyophilization. The lyophilized preparations become ready to use by redissolving them in added distilled water for injection etc. just before use.

### Preparing Dendritic Cells

The invention includes the use of pulsing or administering to antigen presenting cells lysates, mRNA or peptides derived from ValloVax. The antigen presenting cells used in this invention are made by culturing stem cells in an environment that guides the progenitors towards (or promotes outgrowth on the desired cell type. In some instances, differentiation is initiated in a non-specific manner by forming embryoid bodies or culturing with one or more non-specific differentiation factors. Embryoid bodies (EBs) can be made in suspension culture: undifferentiated hPS cells are harvested by brief collagenase digestion, dissociated into clusters or strips of cells, and passaged to non-adherent cell culture plates. The aggregates are fed every few days, and then harvested after a suitable period, typically 4-8 days. Specific recipes for making EB cells from hPS cells can be found in U.S. Pat. No. 6,602,711 (Thomson); WO 01/51616 (Geron Corp.); US 2003/0175954 A1 (Shamblott & Gearhart); and US 2003/0153082 A1 (Bhatia, Robarts Institute). Alternatively, fairly uniform populations of more mature cells can be generated on a solid substrate: US 2002/019046 A1 (Geron Corp.). Maturation of the phagocytic or dendritic cell precursor is achieved in a subsequent step: potentially withdrawing the IL-3, but maintaining the GM-CSF, and adding IL-4 (or IL-13) and a pro-inflammatory cytokine. Other factors that may be helpful at this stage are IL-1 beta, interferon gamma (IFN. gamma.), prostaglandins (such as PGE2), and transforming growth factor beta (TGF beta); along with TNF alpha and/or IL-6 (FIG. 2). A more mature population of dendritic cells should emerge, having some of the characteristics described earlier.

Another embodiment of the present invention provides for a method of producing the composition/vaccine of the present invention and a method of activating the dendritic cells subsequent to administration of ValloVax or derivatives thereof to said dendritic cells. The method comprises providing dendritic cells; culturing the dendritic cells; pulsing the dendritic cells with tumor lysate and at least one TLR ligand. In various embodiments, the dendritic cells may be pulsed with tumor lysate at a concentration of about 50-1000 ug/106-107 PBMDCs, which can be effective at activating PBMDCs in vitro. The dendritic cells may be ones as described above. In a particular embodiment, the dendritic cells may be bone-marrow derived dendritic cells. The TLR ligand may be selected from the group consisting of any suitable components.

According to the above description the method of obtaining the vaccine comprises the following steps:

1. Obtain the tumor-associated vascular antigen: centrifuging, washing, extraction and filtration of the suspension obtained it is possible to obtain the tumor vascular associated antigen.

2. Place distilled and deionized water in a vessel with magnetic stirrer.

3. Centrifuge.

4. Add, one by one or at the same time, the essential and nonessential amino acids to the distilled water in the desired quantities. 5 irradiate at a sufficient dose to achieve mitotic inactivation, said dose in a preferred embodiment 15 Gy.

### Example 1: ValloVax Inhibits Growth of GL-261 Glioma in C57/BL6 Mice

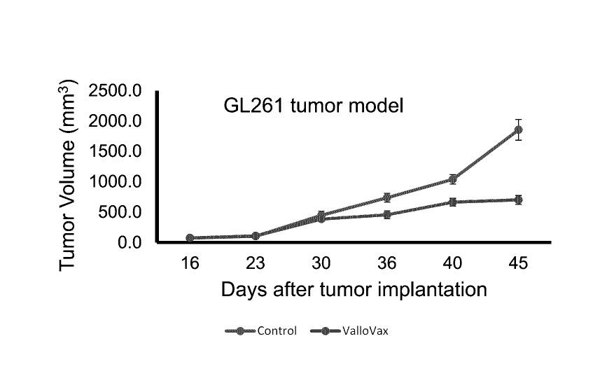

32 C57/BL6 mice were randomized into groups of 16 to receive ValloVax at a concentration of 500,000 cells per mouse, subcutaneously once a week for 4 weeks, or saline control. All mice received an inoculum of 1.7 million GL-261 glioma cells at day 0 of experiment. As shown in FIG. 1, a statistically significant (p<0.05) inhibition of glioma growth as compared to the control was observed.

Having thus described certain embodiments for practicing aspects of the present disclosure, it is to be appreciated that various alterations, modifications, and improvements will readily occur to those skilled in the art. Such alterations, modifications, and improvements are intended to be part of this disclosure, and are intended to be within the spirit and scope of this disclosure.

1. A method of treating glioma comprising the steps of:
a) obtaining a population of endothelial cells;
b) endowing replicative capacity on said endothelial cells;
c) exposing said endothelial cells under conditions resembling the tumor microenvironment;
d) treating said endothelial cells with agents capable of increasing immunogenicity of said endothelial cells; and
e) administering said endothelial cells in a manner to stimulate an immune response against said endothelial cells, as well as an immune response capable of recognizing endothelial cells comprising tumor vascular.

2. The method of claim 1, wherein said endothelial cells are derived from the placenta.

3. The method of claim 2, wherein said placenta is a hemochorial placenta.

4. The method of claim 2, wherein said placenta is term placenta.

5. The method of claim 2, wherein said placenta is a pre-term placenta.

6. The method of claim 2, wherein said placenta is allogeneic to the recipient.

7. The method of claim 2, wherein said endothelial cells are derived from the chorionic portion of the placenta.

8. The method of claim 7, wherein said endothelial cells are derived from the perivascular area of the chorionic portion of the placenta.

9. The method of claim 1, wherein said endothelial cells are generated from a pluripotent stem cell population.

10. The method of claim 9, wherein said pluripotent stem cell population is selected from a group of cells comprising of:
a) embryonic stem cells;
b) inducible pluripotent stem cells;
c) somatic cell nuclear transfer generated stem cells; and
d) parthenogenic stem cells.

11. The method of claim 1, wherein said endothelial cells are generated from endothelial precursor cells.

12. The method of claim 11, wherein said endothelial precursor cells are obtaining from a population of cells selected from a group comprising of:
a) peripheral blood mononuclear cells;
b) adipose tissue derived stromal vascular fraction;
c) umbilical cord blood;
d) perivascular tissue obtained from the wharton's jelly; and
e) perivascular tissue obtained from the omentum.

13. The method of claim 11, wherein said endothelial precursor cells possess expression of the marker kdr-1.

14. The method of claim 1, wherein said endothelial cells possess replicative capacity upon isolation.

15. The method of claim 1, wherein said replicative capacity is endowed by culture in a media containing mitogens.

16. The method of claim 15, wherein said mitogens comprise growth factors.

17. The method of claim 15, wherein said mitogen is fetal calf serum.

18. The method of claim 15, wherein said mitogen is human serum.

19. The method of claim 15, wherein said mitogen is human umbilical cord blood serum.

20. The method of claim 15, wherein said mitogen is platelet lysate.

21.-72. (canceled)
